(19) United States
(12) Patent Application Publication
(10) Pub. No.: US 2013/0157973 A1
(43) Pub. Date: Jun. 20, 2013

(54) PHARMACEUTICAL COMPOSITIONS AND METHODS FOR TREATING GASTROINTESTINAL INFECTIONS AND DISORDERS

(71) Applicant: Gilead Sciences, Inc., Foster City, CA (US)

(72) Inventors: Uri Aryeh Lopatin, San Francisco, CA (US); Daniel B. Tumas, San Carlos, CA (US)

(73) Assignee: Gilead Sciences, Inc., Foster City, CA (US)

(21) Appl. No.: 13/720,968

(22) Filed: Dec. 19, 2012

Related U.S. Application Data
(60) Provisional application No. 61/578,170, filed on Dec. 20, 2011.

(51) Int. Cl. C07D 475/06; A61K 31/522; A61K 45/06; A61K 31/7072; A61K 31/52; C07D 473/18

(52) U.S. Cl. CPC C07D 475/06; A61K 31/52; C07D 473/18; A61K 45/06; A61K 31/7072; A61K 31/522
USPC 514/50; 514/263.2; 514/249; 514/263.37; 514/81

(57) ABSTRACT

Methods of treating gastrointestinal disorders, in a patient in need thereof, including disorders of the liver and pancreas, using an amount of an orally dosed TLR-7 compound in an amount sufficient to increase IFN in the gastrointestinal area, including the liver, but not significantly increasing systemic IFN.

### FIELD OF THE INVENTION

This invention relates to the treatment of disorders of the digestive system, such disorders including allergies, treatment infectious agents and cancer. More particularly, the present invention provides methods and oral dosage forms for increasing interferon expression and interferon concentration that is locally increased in the digestive system, including the intestines, liver and pancreas, but remains systemically low in relation to the locally higher digestive system interferon concentration.

### BACKGROUND OF THE INVENTION

Endogenous Type 1 interferons, such as IFN; α, β, τ, and ω, are secreted largely by plasmacytoid dendritic cells (pDCs), and play a critical role in the recruitment of cells involved in innate immune responses, as well as development of an adaptive immune response. Interferons directly activate macrophage and NK lymphocytes.

Through specific IFN receptors, interferons initiate activation of signal transducer and activator of transcription (STAT) complexes leading to association with Janus Kinase (JAK) and interferon regulatory factor 9 (IRF9), forming an IFN-stimulated gene factor 3 complex, which is translocated to the cell nucleus, binding to specific nucleotide sequences known as IFN-stimulated response elements (ISREs) in the promoters of IFN stimulated genes (ISGs). In this way, interferons initiate a cascade of cytokines that in turn recruit lymphocytes and directly combat infectious agents and tumors.

In addition, Interferons upregulate major histocompatibility complex types I and II (MHC class I and II) and increases the activity of immunoproteosomes in affected cells, for presentation to cytotoxic T cells (MHC class I) and helper T-cells (MHC class II).

Because interferons are capable of initiating pluripotent immune responses, such as facilitation of intercellular communication and inducing the transcription of interferon-stimulated genes (ISGs), the expression of which produces an antiviral state within the cell, they are sometimes given as a primary or adjunctive therapy for the treatment of viral infection or cancer.

For example, hepatitis B virus (HBV) is a deoxyribonucleic acid (DNA) virus that is easily transmissible through perinatal, percutaneous and sexual exposure. Those subjects who develop a chronic HBV infection (CHB) are also at substantial risk of cirrhosis, hepatic decompensation and hepatocellular carcinoma (HCC), which will afflict 15-40% of CHB patients. The availability of a vaccine has reduced the incidence of new HBV infections in the U.S. since the mid 1980's; however, due to immigration from endemic areas in Asia and the Pacific islands, sub-Saharan Africa, the Amazon Basin, and Eastern Europe, the prevalence of CHB remains high, at 0.3-0.5% of the US population. Approximately 4,000 deaths per year result from HBV-related complications in the U.S. alone.

HBV S Antigen (HBsAg) is produced from HBV-infected cells via the replication intermediate: covalently closed circular DNA (cccDNA). The production of HBsAg diverges from that of circulating virus particles and is not directly inhibited by oral antivirals (OAVs). Therefore, the loss of circulating HBsAg may be a marker for the removal of infected cells.

Recent treatment guidelines such as AASLD 2009, EASL 2009 and APASL 2008, acknowledge the importance of HBsAg clearance in CHB. An emerging theme is that HBsAg clearance is associated with definitive remission of the activity of CHB and an improved long term outcome.

Recent data show that the risk of hepatocellular carcinoma (HCC) is lower if HBsAg clearance occurs before 50 years of age. Loss of HBsAg is thus a primary goal of CHB therapy.

Often, because interferon must be administered intravenously, derivatives of interferon are administered, such as PEGylated interferon a, to improve (lower) renal clearance. These interferon preparations include, without limitation, pegylated rIFN-alpha 2b, pegylated rIFN-alpha 2a, rIFN-alpha 2b, rIFN-alpha 2a, consensus IFN alpha (infergen), feron, reaferon, intermax alpha, r-IFN-beta, infergen+actimmune, IFN-omega with DUROS, albuferon, locteron, Albuferon, Rebif, Oral interferon alpha, IFNalpha-2b XL, AVI-005, PEG-Infergen, and Pegylated IFN-beta.

As exogenously administered IFN exerts similar effects, it is mechanistically consistent that IFN has demonstrated substantial therapeutic benefit in patients with chronic HCV and HBV infection. A course of IFN-α/PEG given in HBV, and of IFN-PEG administered with ribavirin to HCV-infected patients, can result in responses equivalent to a clinical cure of the virus in approximately 5% or 40% of treated patients (with HBV and HCV respectively).

Unfortunately, administration of interferons is associated with a constellation of adverse events, including constitutional symptoms (i.e., flu-like symptoms), myelosuppression, elevated liver enzyme levels, and neurologic symptoms, which to some extent affect the majority of patients.

Interferons are themselves stimulated in vivo by pattern recognition receptor proteins, such as toll-like receptors (TLR). There are eleven known TLRs in man. These pattern recognition receptors are activated by pathogen-associated molecular patterns (PAMPs), for example, conserved microbial motifs such as peptidoglycan (TLR2), CpG DNA (TLR9), viral RNA (TLR3/7/8), bacterial flagellin (TLR5) and lipopolysacharide (LPS) associated with Gram negative bacteria (TLR4). TLR1 and TLR6 form heterodimers with TLR2, and act to stabilize TLR2. TLRs are present in pDCs, where they assist in the sentry role of these cells.

Because TLRs can initiate an interferon response in patients, one treatment strategy has been to develop agonists of relevant TLRs to provide an alternative to IFN administration. Unfortunately, many of the side effects inherent in IFN therapy are also found in patients after TLR agonist administration.

Therefore, it would be desirable to provide a method of treating diseases and conditions associated with improvement with interferon therapy, without inducing the unwanted side effects associated with interferon therapy.

Some attempts have been made to avoid the side effects associated with systemic interferon therapy. For example, the imiquimod, 3-(2-methylpropyl)-3,5,8-triazatricyclo[7.4.0.02,6]trideca-1(9),2(6),4,7,10,12-hexaen-7-amine, is formulated for topical use on the skin More recently, the compounds SM-324405 and AZ12441970, both from AstraZeneca, are formulated for aerosol inhalation for the treatment of asthma, were developed as antedrugs, having ester groups that are rapidly cleaved in plasma to reduce systemic exposure.

There is a need for an orally available. TLR agonist for treating gastrointestinal disorders, including liver disorders.

### SUMMARY OF THE INVENTION

It has now been discovered that providing a presystemic oral dose of an orally available TLR agonist compound will lead to localized induction of IFN in the gastrointestinal system, particularly in the intestines, pancreas and liver, without inducing significant systemic IFN in a patient in need thereof.

Thus, there is provided a method of treating a gastrointestinal disorder in a human patient in need thereof, comprising administering to the patient an orally administered amount of a TLR modulator sufficient to provide modified IFN expression in the gastrointestinal area, but in an amount less than sufficient to significantly alter systemic IFN.

In one embodiment of the invention, there is provided a method of treating a gastrointestinal disorder, including a disorder of the liver or pancreas, comprising as a modality of treatment wherein a TLR-7 agonist compound having the formula:
4-amino-2-butoxy-8-(3-(pyrrolidin-1-ylmethyl)benzyl)-7,8-dihydropteridin-6(5H)-one

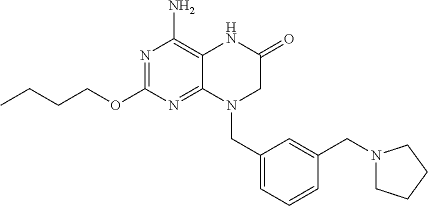 [SMILES: CCCCOc1nc(N)c2c(n1)N(Cc1cccc(CN3CCCC3)c1)CC(=O)N2]

is administered to a patient in need thereof, in a total dose of less than 12 mg per day.

In another embodiment, a TLR-7 agonist compound having the formula:
4-amino-2-butoxy-8-(3-(pyrrolidin-1-ylmethyl)benzyl)-7,8-dihydropteridin-6(5H)-one

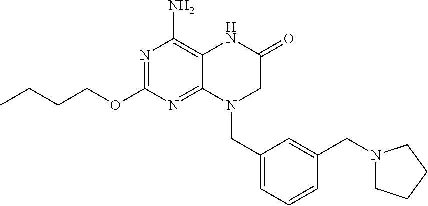 [SMILES: CCCCOc1nc(N)c2c(n1)N(Cc1cccc(CN3CCCC3)c1)CC(=O)N2]

is administered to a patient in need thereof, in a total dose of less than 12 mg every other day.

In another embodiment, a TLR-7 agonist compound having the formula:
4-amino-2-butoxy-8-(3-(pyrrolidin-1-ylmethyl)benzyl)-7,8-dihydropteridin-6(5H)-one

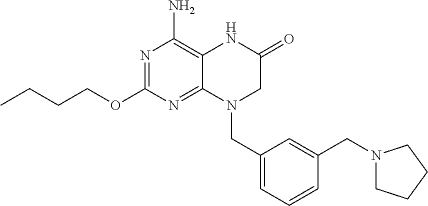 [SMILES: CCCCOc1nc(N)c2c(n1)N(Cc1cccc(CN3CCCC3)c1)CC(=O)N2]

is administered to a patient in need thereof, in a total dose of less than 12 mg twice per week.

In another embodiment, a TLR-7 agonist compound having the formula:
4-amino-2-butoxy-8-(3-(pyrrolidin-1-ylmethyl)benzyl)-7,8-dihydropteridin-6(5H)-one

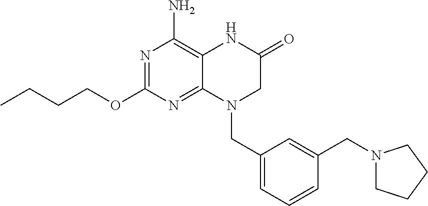 [SMILES: CCCCOc1nc(N)c2c(n1)N(Cc1cccc(CN3CCCC3)c1)CC(=O)N2]

is administered to a patient in need thereof in a total dose of less than 12 mg once per week.
I. Treatment of Diseases and Conditions with the Present Invention

A variety of diseases and disorders are treatable with the methods and compositions of the present invention.

For example, viral diseases of the liver, such as hepatitis A, hepatitis B, hepatitis C, or hepatitis D, solid tumors such as hepatocellular carcinoma (HCC),

Allergic and autoimmune disorders in the gastrointestinal system would be amenable to treatment, such as Crohns disease, graft vs. host disease, gastrointestinal organ transplant, including liver and pancreas transplant, and food allergies, including peanut allergies.

These disorders are merely exemplary.

### Exemplary Compounds

A) A compound of structural formula:

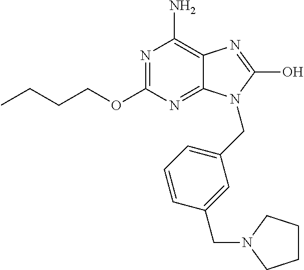 [SMILES: CCCCOc1nc(N)c2nc(O)n(Cc3cccc(CN4CCCC4)c3)c2n1]

6-amino-2-butoxy-9-(3-(pyrrolidin-1-ylmethyl)benzyl)-9H-purin-8-ol;

B) A compound of structural formula:

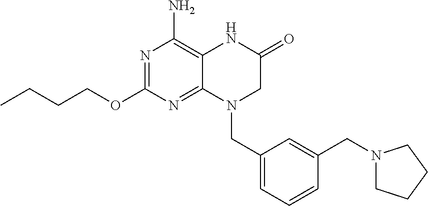 [SMILES: CCCCOc1nc(N)c2c(n1)N(Cc1cccc(CN3CCCC3)c1)CC(=O)N2]

4-amino-2-butoxy-8-(3-(pyrrolidin-1-ylmethyl)benzyl)-7,8-dihydropteridin-6(5H)-one;

Compound A is a TLR-7 agonist. Compound A, and methods to make it are disclosed in U.S. Published patent application US2008/007955.

Compound B is also a TLR-7 agonist. Compound B, and methods to make it are disclosed in U.S. Published patent application US2010/0143301.

### DETAILED DESCRIPTION OF THE INVENTION

### I. Definitions

AE adverse event
ALT alanine aminotransferase
AST aspartate aminotransferase
BLQ below limit of quantitation
DAA direct-acting antiviral
DNA complementary deoxyribonucleic acid
DLT dose-limiting toxicity
GALT gut-associated lymphoid tissue
GGT gamma-glutamyltransferase
HBV hepatitis B virus
HCC hepatocellular carcinoma
HCV hepatitis C virus
HED human equivalent dose
IFE initial food effect
IFN-α interferon-α
IND Investigational New Drug Application
ISG interferon-stimulated gene
IV intravenous
N/A Not Applicable
ND not determined
NOAEL no observed adverse effect-level
PBMC peripheral blood mononuclear cells
PEG pegylated interferon
Peg-IFN-alfa-2a peginterferon alfa 2a
Peg-IFN-alfa-2b peginterferon alfa 2b
PD
pDC
Pharmacodynamic
plasmacytoid dendritic cells
PK pharmacokinetic
QOD every other day
RBV ribavirin
RNA ribonucleic acid
S/MAD single/multiple ascending dose
TLR-7 toll-like receptor-7
WHV woodchuck hepatitis virus

### Pharmacokinetic Abbreviations

AUC Area under the concentration versus time curve
AUCinf Area under the concentration versus time curve extrapolated to infinite time, calculated as
AUClast+(Clastaz)
AUClast
AUCtau

### Salt Forms of the Compounds of the Present Invention

Typically, but not absolutely, the salts of the present invention are pharmaceutically acceptable salts. Salts encompassed within the term “pharmaceutically acceptable salts” refer to non-toxic salts of the compounds of this invention.

Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates. Thus, where the term “a pharmaceutically acceptable salt, solvate, tautomer, or prodrug thereof” is used, it is to be appreciated that each of these forms is independent of the others, and also includes combinations thereof. For example, the term “a pharmaceutically acceptable salt, solvate, tautomer, or prodrug thereof” includes, without limitation, a pharmaceutically acceptable salt alone, two or more pharmaceutically acceptable salts together, a pharmaceutically acceptable salt and prodrug, a pharmaceutically acceptable salt of a prodrug, and a pharmaceutically acceptable salt which is a solvate, for example. In the case of tautomers, when tautomerization is possible in a compound, a given illustrative chemical structure, even when illustrating only one form, is to be interpreted as including its tautomeric structural form as well.

### Pharmaceutical Formulations

The compounds of this invention are typically formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986), herein incorporated by reference in its entirety. Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10.

While it is possible for the active ingredients to be administered alone it may be preferable to present them as pharmaceutical formulations. The formulations of the invention, both for veterinary and for human use, comprise at least one active ingredient, together with one or more acceptable carriers and optionally other therapeutic ingredients. The carrier is “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.), herein incorporated by reference in its entirety. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient.

Pharmaceutical formulations according to the present invention comprise one or more compounds of the invention together with one or more pharmaceutically acceptable carriers or excipients and optionally other therapeutic agents. Pharmaceutical formulations containing the active ingredient may be in any form suitable for the intended method of administration. Tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Tablets may be formed with Compound A or Compound B as an active ingredient, and may be dosed as 0.1-mg, 0.5-mg, 1-mg, 2-mg, and 5-mg strength tablets. The tablets may contain commonly used excipients including lactose anhydrous, microcrystalline cellulose, croscarmellose sodium, magnesium stearate, polyethylene glycol, polyvinyl alcohol, talc, and titanium dioxide

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth herein, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The amount of active ingredient that may be combined with the carrier material to produce a single dosage form will vary depending upon the host treated and the particular mode of administration. For example, a time-release formulation intended for oral administration to humans may contain approximately 0.5 to 12 mg of active material compounded with an appropriate and convenient amount of carrier material which may vary from about 1 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations for rectal administration may be presented as a suppository with a suitable base comprising for example cocoa butter or a salicylate.

Compounds of the invention can also be formulated to provide controlled release of the active ingredient to allow less frequent dosing or to improve the pharmacokinetic or toxicity profile of the active ingredient. Accordingly, the invention also provided compositions comprising one or more compounds of the invention formulated for sustained or controlled release.

### Combination Therapy

In another embodiment, the compounds of the present invention may be combined with one or more active agent.

Combinations for the treatment of hepatitis B with compound A or compound B include nucleoside reverse transcriptase inhibitors; non-nucleoside reverse transcriptase inhibitors; protease inhibitors; cyclophilin inhibitors; immune modulators; and combinations thereof.

Exemplary combination products for treatment of hepatitis B with compound A or compound B include: etbecavir, telbivudine, lamisvudine, adofovir dipivoxil, entecavir, tenofovir disoproxil fumarate, emtricitabine, tenofovir dipivoxil and its salts and co-crystals; and yeast-based therapeutic vaccinations, such as Tarmogens®, from GlobeImmune, inc.; and combinations thereof.

Combinations for treatment of hepatitis C with compound A or compound B include: Nucleoside or nucleotide inhibitors of HCV NS5B polymerase; non-nucleoside inhibitors of HCV NS5B polymerase, HCV NS5A inhibitors; HCV NS3 protease inhibitors; HCV NS4B protease cofactor inhibitors; cyclophilin inhibitors; HCV internal ribosome entry site (IRES) inhibitors; and combinations thereof.

Exemplary combination active ingredients for treatment of hepatitis C with compound A or compound B include: ribavirin; sofosbuvir; declatasvir; tegobuvir; boceprevir; telaprevir; GS-5885 (NS5A inhibitor); GS-9451 (protease inhibitor); GS-5816 (protease inhibitor); MK-5172 (protease inhibitor); filibuvir; GS-9857 (protease inhibitor); GS-9669 (non-nucleoside polymerase inhibitor); ABT-450 (protease inhibitor); ABT-450 with ritonavir; ABT-333 (polymerase inhibitor); ABT-267 (NS5A inhibitor); and combinations thereof.

Combinations for the treatment of HIV with compound A or compound B include: Entry inhibitors; capsid inhibitors; nucleoside reverse transcriptase inhibitors (NRTI); non-nucleoside reverse transcriptase inhibitors (NNRTI); protease inhibitors (PI); integrase inhibitors; maturation inhibitors; and combinations thereof.

Exemplary combination products for treatment of HIV with compound A or compound B include: Maraviroc (Selzentry®); enfuvirtide (Fuzeon®); tenofovir disoproxil fumarate with emtricitabine (Truvada®); tenofavir disoproxil fumarate with emtricitabine and efavirenz (Atripla®); elvitegravir with emtricitabine, cobisistat and tenofavir disoproxil fumarate (Stribild®); lamivudine with zidovudine (Combivir®); abacavir with zidovudine and lamivudine (Trizivir®); lopinavir with ritonovir (Kaletra®); abacavir with lamivudine (Epzicom®—United States, Kivexa®—Europe), rilpivarine with tenofavir disoproxil fumarate and emtricitabine (Complera®); elvitegravir with emtricitabine, cobisistat and tenofovir dipivoxil and its salts and co-crystals; and combinations thereof.

In yet another embodiment, there is disclosed a pharmaceutical compositions comprising a compound of the present invention, or a pharmaceutically acceptable salt thereof, in combination with at least one additional active agent, and a pharmaceutically acceptable carrier or excipient. In yet another embodiment, the present application provides a combination pharmaceutical agent with two or more therapeutic agents in a unitary dosage form. Thus, it is also possible to combine any compound of the invention with one or more other active agents in a unitary dosage form.

The combination therapy may be administered as a simultaneous or sequential regimen. When administered sequentially, the combination may be administered in two or more administrations.

Co-administration of a compound of the invention with one or more other active agents generally refers to simultaneous or sequential administration of a compound of the invention and one or more other active agents, such that therapeutically effective amounts of the compound of the invention and one or more other active agents are both present in the body of the patient.

Co-administration includes administration of unit dosages of the compounds of the invention before or after administration of unit dosages of one or more other active agents, for example, administration of the compounds of the invention within seconds, minutes, or hours of the administration of one or more other active agents. For example, a unit dose of a compound of the invention can be administered first, followed within seconds or minutes by administration of a unit dose of one or more other active agents. Alternatively, a unit dose of one or more other active agents can be administered first, followed by administration of a unit dose of a compound of the invention within seconds or minutes. In some cases, it may be desirable to administer a unit dose of a compound of the invention first, followed, after a period of hours (e.g., 1-12 hours), by administration of a unit dose of one or more other active agents. In other cases, it may be desirable to administer a unit dose of one or more other active agents first, followed, after a period of hours (e.g., 1-12 hours), by administration of a unit dose of a compound of the invention.

The combination therapy may provide “synergy” and “synergistic effect”, i.e. the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g., in separate tablets, pills or capsules, or by different injections in separate syringes. In general, during alternation therapy, an effective dosage of each active ingredient is administered sequentially, i.e. serially, whereas in combination therapy, effective dosages of two or more active ingredients are administered together.

### Kits

In another embodiment, a kit comprising a course of treatment, with or without instructions for use, is provided. For example, a kit comprising an oral dosage form pharmaceutical composition of compound B, in a package adapted for distribution of said oral dosage form pharmaceutical composition in an amount between 0.5 mg and 12 mg. once per week. Such a kit typically provides a sequential series of solid dosage form tablets or capsules, provided in a structure adapted to provide a total daily, twice weekly or weekly dose of less than 12 mg. of compound A or compound B per day, over the course of a week or a month, for example. Alternatively, a kit is provided that contains multiple solid, oral dosage form tablets or capsules in a dispenser, said dispenser including a reminding device. The reminding device may be in the form of a calendar, or may provide an audible signal for reminding a patient that the oral dosage form composition should be taken at a predetermined interval, such as once or twice per week.

In one embodiment of the kit, a blister pack is provided with a single dose of less than 12 mg. of compound A or B in one section of the blister pack, with inactive ingredient tablets in the remaining sections of the blister pack. For example, a weekly dosage pack may contain a tablet containing a single dose of less than 12 mg. of compound B in one blister section, with six additional blister sections containing tablets with no active ingredients.

In the case of single dosage form combination products formulated with an appropriate active ingredient other than compound A or compound B together with compound A or compound B, the blister pack may contain seven sections per week, with a single tablet containing both compound A or compound B together with the additional active ingredient or ingredients, and the remaining six sections of the weekly regimen blister pack containing tablets with only active ingredient or ingredients other than compound A or compound B.

For example, a four week regimen blister pack may contain a series of four, seven day sections, with a first section comprising a solid, orally available dosage form with a combination of compound B and one or more additional active ingredients, and the remaining six sections containing a solid, orally available dosage form with a combination of the active ingredients without compound B.

### Biological Data

Referring to FIG. 1, plasmacytoid dendritic cells present in the gut and/or liver are activated by local exposure to an orally available TLR agonist compound to produce IFN-α and stimulate ISG induction in lymphocytes and other cells as they circulate through the GALT and liver. ISG induction may occur in the liver by a similar effect (through either local IFN-α produced from stimulated pDCs residing in the liver or from a first pass effect on the liver from portal blood IFN-α produced from pDCs in the GALT). ISGs produced by IFN-α can mediate antiviral effects. As a consequence of the presystemic stimulation of TLR-7, local ISGs and other effectors of an interferon-mediated antiviral response may occur at reduced oral doses of an orally available TLR agonist compound that do not cause induction of serum/systemic IFN-α or clinical signs (increased body temperature and heart rate).

Presystemic (local) induction of an innate immune response can be detected noninvasively by at least 2 methods. In healthy subjects, the level of ISG induction in circulating blood cells reflects exposure of white blood cells trafficking through the GALT and the liver with exposure to an interferon-rich environment. Additionally, in subjects with viral hepatitis, increases in local interferon production may be detected by a reduction in serum viremia.

### Pharmacokinetics and Pharmacodynamics

Table 1 and Table 2 present the PK parameters of Compound B following the administration of a single dose of Compound B in the fasted cohorts and fed cohorts, respectively. Mean maximal plasma concentration values (Cmax) were higher with increasing dose in the fasted treatment groups. Mean maximal plasma concentration values were lower when Compound B (8 mg) was administered with moderate- or high-fat meal or following a high fat-meal than when Compound B was administered under fasting conditions. Similarly, mean AUC values in the fed cohorts were 47% to 73% of those values in the fasted cohorts; the lowest exposures were observed when Compound B 8 mg was coadministered with a moderate-fat meal. Median terminal Compound B half-life values ranged from 14.65 to 26.92 hours except for the 0.3-mg group for which plasma concentrations were measured only to 24 hours postdose.

TABLE 1
Compound B Pharmacokinetic Parameters
Following Administration of a Single Dose of Compound B by
Treatment (Pharmacokinetic Analysis Set)

| | Cohort 1 | Cohort 2 | Cohort 3 | Cohort 4 | Cohort 5 | Cohort 6 | Cohort 7 |
|---|---|---|---|---|---|---|---|
| COMPOUND B | 0.3 mg | 1 mg | 2 mg | 4 mg | 6 mg | 8 mg | 12 mg |
| PK PARAMETER | (N = 6) | (N = 6) | (N = 6) | (N = 6) | (N = 6) | (N = 6) | (N = 6) |
| Cmax (pg/mL) | 184.2 | 440.1 | 633.2 | 2928.7 | 7261.2 | 8335.6 | 11,968.9 |
| Mean (% CV) | (75.5) | (59.0) | (88.9) | (42.9) | (71.7) | (51.7) | (12.3) |
| Tmax (h) | 3.00 | 3.00 | 6.00 | 4.00 | 2.50 | 2.51 | 1.51 |
| Median (Q1, | (2.00, | (1.00, | (4.00, | (2.00, | (2.00, | (2.00, | (1.00, |
| Q3) | 4.00) | 6.00) | 6.00) | 4.00) | 3.00) | 4.00) | 2.00) |
| AUCinf (pg · h/mL) | 2969.4 | 9231.0 | 11,267.9 | 57,179.6 | 76,864.8 | 109,110.3 | 140,368.7 |
| Mean (% CV) | (86.2)a | (29.7) | (48.0) | (45.7) | (64.0) | (73.9) | (54.4) |
| T½ (h) | 10.38 | 26.92 | 24.30 | 17.16 | 14.65 | 19.54 | 16.29 |
| Median (Q1, Q3) | (6.66, | (18.62, | (19.64, | (15.00, | (12.52, | (14.72, | (15.33, |
| | 21.52)^{a} | 29.09) | 26.72) | 26.07) | 17.33) | 22.16) | 20.16) |

Note:
48-hour plasma PK sample was not drawn for subjects enrolled in Cohort 1.
^{a}The 0.3-mg group had limited data available data during the terminal elimination phase relative to the long half-life for Compound B, and high intersubject variability was observed in that cohort. These values should be interpreted with caution.

TABLE 2
Compound B Pharmacokinetic Parameters Following Administration of a Single Dose of
Compound B Fasted, With a Moderate-fat Meal, With a High-fat Meal, and Following a
High-fat Meal (4 Hours) (Pharmacokinetic Analysis Set)

| | | | Cohort 8 | |
|---|---|---|---|---|
| | | IFE Cohort | 8 mg with a | Cohort 9 |
| | Cohort 6 | 8 mg with a | moderate-fat | 8 mg post |
| COMPOUND B | 8 mg fasted | high-fat meal | meal | high-fat meal |
| PK PARAMETER | (N = 6) | (N = 6) | (N = 6) | (N = 6) |
| Cmax (pg/mL) | 8335.6 | 5238.7 | 2040.6 | 4532.8 |
| Mean (% CV) | (51.7) | (85.3) | (42.6) | (54.5) |
| Tmax (h) | 2.51 | 3.00 | 2.00 | 5.00 |
| Median (Q1, Q3) | (2.00, 4.00) | (3.00, 3.00) | (1.00, 3.00) | (4.00, 6.00) |
| AUCinf (pg · h/mL) | 109,110.3 | 71,433.3 | 51,089.2 | 79,533.9 |
| Mean (% CV) | (73.9) | (55.1) | (41.8) | (44.3) |
| T½ (h) | 19.54 | 23.55 | 21.64 | 20.54 |
| Median (Q1, Q3) | (14.72, 22.16) | (19.86, 28.51) | (16.42, 27.32) | (16.22, 29.53) |

IFE, initial food effect

In humans, Compound B signals through both the Toll-like receptor (TLR) 7 and 8 pathways, inducing cytokines including IFN-α, interleukin (IL)-12 and tumor necrosis factor alpha (TNF-α) from innate immune cells

Two randomized, double-blind phase IIa studies of Compound B administered two times per week for 4 weeks. Multicenter study (U.S.): 12 subjects received Compound B 0.01 mg/kg and 4 received placebo. Single center study (France): 6 subjects received 0.01 mg/kg, 11 received 0.02 mg/kg and 6 received placebo.

### Results

Compound B 0.01 mg/kg was tolerated; two 0.2 mg/kg subjects discontinued treatment. More subjects reported severe grade adverse events at 0.02 mg/kg; events were consistent with systemic cytokine induction, including fever, headache, shivering, and lymphopenia. Mean maximum serum Compound B concentrations were 3.82±1.47 and 7.55±4.17 ng/mL for 0.01 mg/kg and 0.02 mg/kg, respectively. At 0.02 mg/kg, two, three and one subjects had maximal reductions in viral levels of at least 1-, 2- and 3-logs, respectively; reductions were generally transient.
Interferon-alpha levels appeared correlated with decreases in viral titer and lymphocyte counts, as well as increase in neutrophil counts.

### Conclusions

Oral administration of Compound B 0.02 mg/kg transiently reduced viral levels but was associated with adverse effects similar to interferon-alpha.

In a placebo-controlled, single administration study in 48 healthy adults of up to 0.05 mg/kg, the maximum tolerated oral dose of Compound B was 0.03 mg/kg; in a placebo-controlled, multiple administration study in 25 healthy adults, the maximum administered regimen of 0.2 mg/kg two times per week for 2 weeks followed by 0.03 mg/kg two times per week for 2 weeks was adequately tolerated.

For both studies, major inclusion criteria were males or females 18-70 years of age who had evidence of chronic HCV infection with all of the following: positive HCV serology by enzyme-linked immunosorbent assay, serum HCV RNA>10,000 copies/mL, elevated serum alanine aminotransferase (ALT) level within 6 months, and a liver biopsy within 24 months demonstrating changes consistent with HCV infection. Exclusion criteria included clinically meaningful cirrhosis on prior liver biopsy (U.S. study), positive serology for possible autoimmune hepatitis (ANA˜1:640, ASMA>1:320, ALKM antibody>1:320), hepatocellular neoplasia, anemia (<12 g/dL for men, <11 for women), thrombocytopenia (<90,000/μL), leukopenia (<2500 cells/μL), neutropenia (<1500 cells/μL, U.S. study), ALT>1000 U/L (French study) or aspartate aminotransferase (AST) or ALT>500 U/L (U.S. study), bilirubin>1 mg/dL, decompensated liver disease, other liver diseases, positive serology for HIV, positive HBsAg, prior organ transplantation, significant psychiatric disease, alcohol or drug abuse within 12 months, systemic immunomodulatory or investigational therapy within 3 months, and significant cardiac, pulmonary, systemic inflammatory or thyroid disease.

For both studies, treatment assignment within each cohort (16 subjects U.S. study; 8 subjects French study) was determined via computer-generated randomization. In the U.S. study, subjects were assigned centrally across centers. Active to placebo assignment was 3:1 for each cohort. Sample sizes were not prospectively powered.

### 2.2. Study Design

All subjects were to receive study drug two times per week for 4 weeks. Subjects self-administered study drug at home except on study visits with pharmacokinetic and pharmacodynamic sampling where it was administered in the clinic. Compound B or matching placebo was administered as oral capsules (3M Pharmaceuticals, Saint Paul, Minn.). In the U.S. study, subjects received 0.01 mg/kg of resiquimod. In the French study, sequential cohorts were to have received 0.01, 0.02 and 0.03 mg/kg (due to an adverse event, this dose level was not enrolled, see Results) of Compound B per dose, respectively; a safety review was performed prior to escalation.

### Pharmacodynamics

Serum HCV RNA was measured by quantitative polymerase chain reaction (NGI). Subjects were categorized as responders (reduction from baseline of ˜2 logs) or non-responders at the end-of-treatment visit (day 29), and at the last follow-up visit (day 113 U.S. study, and day 57 French study).

Samples for cytokines were obtained at 0, 2, 4, 6, 8, 12 and 24 h after the first dose, prior to dosing at days 8, 15, 22 or 25 (see above regarding pharmacokinetics) and day 29. Serum IL-6, IL-1RA, TNF-α and IFN-γ were measured by enzyme-linked immunosorbent assay (Immunotech, Cedex, France) and neopterin by immunoenzymatic assay (Immunotech, Cedex, France). Serum type I IFN levels were determined by bioassay [16]. Serum 2′,5′ oligoadenylate synthetase (2′5′ AS) was measured by radioimmunoassay (Eiken Chemical Co. Ltd., Tokyo, Japan). Serum IL-12 p40 was measured by enzyme-linked immunosorbent assay (R&D Systems, Minneapolis, Minn.) Immunophenotyping of T lymphocytes in the U.S. study was performed at NGI.

### Results

In both studies, there were no clinically meaningful changes in physical examinations. A dose-dependent initial increase in absolute neutrophil count (ANC) and decrease in absolute lymphocyte count were observed post-dose (Table 3); ANC subsequently appeared decreased, overall, in the Compound B groups (Table 3). Subjects in the 0.02 mg/kg group had greater maximum grade ANC and ALC toxicity (at any time during treatment period) by Common Terminology Criteria for Adverse Events (CTCAE) (Table 3). Of the 9 subjects with severe pyrexia, 6 had grade 3 and 2 had grade 4 ALC decrease, and 2 each had grade 2 and grade 3 ANC decrease.

TABLE 3
Change from baseline in absolute neutrophil and lymphocyte counts

| | Change in absolute neutrophil count | | | Change in absolute lymphocyte count | | |
|---|---|---|---|---|---|---|
| | (cells/mm3) | | | (cells/mm3) | | |
| Combined studies | Placebo | 0.01 mg/kg | 0.02 mg/kg | Placebo | 0.01 mg/kg | 0.02 mg/kg |

Day 1 (8 h)

| | | | | | | |
|---|---|---|---|---|---|---|
| N | 10 | 18 | 11 | 10 | 18 | 11 |
| Median | 480 | 677 | 3170 | 174 | −595 | −1720 |
| (range) | (54, 3130) | (−970, 5200) | (−2270, 6460) | (−600, 650) | (−1595, 508) | (−3080, −50) |
| Mean ± SD | 760 ± 881 | 804 ± 1312 | 2875 ± 2411 | 123 ± 364 | −638 ± 641 | −1638 ± 774 |

Day 1 (24 h)

| | | | | | | |
|---|---|---|---|---|---|---|
| N | 4a | 11a | 6a | 4a | 11a | 6a |
| Median | 43 | −533 | −650 | −232 | −148 | −520 |
| (range) | (−326, 788) | (−1098, 1667) | (−4530, 1700) | (−416, −65) | (−755, 656) | (−2550, −250) |
| Mean ± SD | 137 ± 546 | −152 ± 781 | −878 ± 2148 | −236 ± 162 | −156 ± 398 | −965 ± 892 |

Day 29 end-of-treatment visit

| | | | | | | |
|---|---|---|---|---|---|---|
| N | 10 | 18 | 9b | 10 | 18 | 9b |
| Median | −166 | −65 | −470 | −251 | −153 | −295 |
| (range) | (−1060, 980) | (−3680, 491) | (−3790, 1760) | (−930, 340) | (−1440, 969) | (−2040, 250) |
| Mean ± SD | −54 ± 549 | −452 ± 999 | −727 ± 1647 | 271 ± 385 | −166 ± 506 | −478 ± 620 |

Maximum decrease during treatment period

| N | 10 | 18 | 11 | 10 | 18 | 11 |
|---|---|---|---|---|---|---|
| Median | −328 | −738 | −730 | −550 | −865 | −1796 |
| (range) | (−1244, 300) | (−4780, −15) | (−4530, 360) | (−1228, −10) | (−1595, 0) | (−3080, −310) |
| Mean ± SD | −374 ± 463 | 966 ± 1078 | −1359 ± 1370 | −530 ± 370 | −889 ± 510 | −1670 ± 723 |
| Maximum | | | | | | |
| toxicity | Neutropeniac (subject N, %) | | | Lymphopeniad (subject N, %) | | |
| Grade 1 | 3 (30%) | 5 (28%) | 1 (9%) | 1 (10%) | 1 (6%) | 2 (18%) |
| Grade 2 | 2 (20%) | 5 (28%) | 3 (27%) | 0 | 1 (6%) | 0 |
| Grade 3 | 1 (10%) | 0 | 2 (18%) | 0 | 0 | 6 (55%) |
| Grade 4 | 0 | 0 | 0 | 0 | 0 | 2 (18%) |

aNot all subjects in French study had sampling at 24 h post dosing Day 1. One subject in U.S. study missing sample.
bTwo subjects discontinued treatment prior to day 29 end-of-treatment visit.
c ANC grade 1 < lower limits of normal to 1500, grade 2 < 1500-1000, grade 3 < 1000-500, grade 4 < 500 cells/μL. Lower limits of normal 2250 cells/mm3 for U.S. and 1700 cells/mm3 for French study.
d ALC grade 1 < lower limits of normal to 800, grade 2 < 800-500, grade 3 < 500-200, grade 4 < 200 cells/μL. Lower limits of normal 675 cells/mm3 for U.S. and 1200 cells/mm3 for French study.

Neither ALT nor AST levels appeared to be affected in the U.S. study (data not shown). In the French study, the proportion of subjects with AST and ALT elevations decreased slightly from day 1 to day 29 in the 0.02 mg/kg group, 55% (6/11) to 11% (1/9) and 82% (9/11) to 56% (5/9), respectively.

### Pharmacokinetics

Compound B concentrations after single (Table 4) and multiple dosing rose rapidly, reaching Cmax between 0.5 and 2.0 h post-dose. Thereafter, Compound B concentrations appeared to decline in a biphasic manner, the terminal phase becoming apparent between 8 and 16 h post-dose. With dose doubling there was almost a 2-fold increase in both mean serum Compound B Cmax and area under the curve (AUC), suggesting linear kinetics within the dose range studied (Table 4). There was little or no evidence of drug accumulation on repeat dosing as determined by drug levels measured on day 22/25 for the U.S. study or day 15 for the French study (data not shown). Large inter-subject variability was observed, with day 1 coefficients of variance for Cmax of 39% and 44% for 0.01 mg/kg (U.S. study and French study, respectively) and 55% for 0.02 mg/kg. Despite the large inter-subject variability, little intra-subject variability was observed for Cmax or AUC; comparable values were obtained after single and repeated administration for a subject (data not shown). The two 0.02 mg/kg subjects who discontinued treatment for severe grade lymphopenia and for severe grade flu-like symptoms had Compound B Cmax values of 12.7 and 10.8 ng/mL, respectively.

TABLE 4
Pharmacokinetic parameters of Compound B following oral
administration, first dose, studies combined

| | 0.01 mg/kg | 0.02 mg/kg |
|---|---|---|
| Total subjects | 12 | 11 |
| Tmaxa (h) | 1.0 | 1.0 |
| Cmaxb (ng/mL) | 3.82 ± 1.47 | 7.55 ± 4.17 |
| AUCc (ng h/mL) | 20.97 ± 13.65 | 45.66 ± 43.98 |
| T½, z (h) | 6.77 ± 3.10 | 6.82 ± 3.51 |
| CL/F (L/h/kg) | 0.57 ± 0.42 | 1.11 ± 1.54 |
| Vz/F (L/kg) | 4.29 ± 1.84 | 6.58 ± 3.83 |

T½, z: terminal phase half-life. Ln2 divided by apparent terminal phase rate constant estimated by log linear regression of at least three data concentration-time points after Tmax. Results reported as means ± standard deviation.
CL/F: apparent clearance. Results reported as means ± standard deviation.
Vz/F: apparent volume of distribution. Results reported as means ± standard deviation.
aTmax: time of maximum drug concentration, determined by direct inspection of the drug concentration versus time data point values. Results reported as median.
bCmax: maximum observed drug concentration, determined by direct inspection of the drug concentration versus time data point values. Results reported as means ± standard deviation.
cAUC: area under the curve concentration versus time curve extrapolated to infinity, calculated by extrapolation of the elimination slope from tz to infinity (tz = time point for last sample on pharmacokinetic profile with quantifiable drug). Results reported as means ± standard deviation.

### Pharmacodynamics

After the first dose, there appeared to be a dose-dependent decrease in serum HCV RNA levels peaking at about 24 h and trending toward baseline by 48 h (FIG. 1). One, five and six subjects had at least a 1-log reduction in HCV levels at any time during the study for the placebo, Compound B 0.01 and 0.02 mg/kg groups, respectively (FIG. 2). Two, three and one subjects in the 0.02 mg/kg group had maximal decreases of at least 1-, 2- and 3-logs, respectively (FIG. 2). Of the 11 Compound B subjects with at least a 1-log reduction at anytime during, the study, the HCV Rmax occurred within 48 h after dose 1 in 6 subjects, and at day 29 or after in 5 subjects. At end-of-treatment visit only one subject (0.02 mg/kg) was considered a responder per protocol (□2 log reduction); this was not sustained on follow-up.

There appeared to be a possible relationship between Compound B Cmax and Rmax after dose 1 for HCV RNA (adjusted R2 0.4833, Spearman correlation coefficient 0.51503, p<0.0008), IFN-γ (0.5940, 0.6196, <0.0001), IL-1RA (0.6350, 0.7698, <0.0001), IFN-α (0.5118, 0.6354, <0.0001) and NPT (0.5301, 0.68610, <0.0001; FIGS. 3a-c). IFN-α Rmax appeared to be associated with HCV Rmax (adjusted R2 0.4944, Spearman correlation coefficient 0.6204, p<0.0001; FIG. 3d) and 8 h change ALC (0.7369, −0.7495, <0.0001) and possibly within 8 h change in ANC (0.3628, 0.4598, 0.0032; FIG. 3d) Median IFN-α Rmax after dose 1 appeared to be higher in those subjects who had a maximum CTCAE grade of 3 for ANC, 3 or 4 for ALC, and who had severe pyrexia (FIG. 3f). The two Compound B 0.02 mg/kg subjects who discontinued treatment for severe grade lymphopenia and for severe grade flu-like symptoms had IFN-α Rmax post-dose 1 of 15,557 and 3946 IU/mL, respectively. There did not appear to be evidence of a relationship between Compound B Cmax and the Rmax after the dose 1 with IL-6 (adjusted R2 0.1280, Spearman correlation coefficient 0.4023, p<0.0111), IL-12 p40 (0.0156, 0.2062, 0.2079), 2′5′ AS (0.0194, 0.1945, 0.2354) and TNF-α (−0.0244, 0.0989, 0.5491). Clinically relevant changes in CD4+ lymphocyte counts or CD4+/CD8+ lymphocyte ratios were not observed.

What is claimed is:

1. A method of treating a gastrointestinal disorder in a human patient in need thereof, comprising administering to the patient an orally administered amount of a TLR agonist sufficient to provide modified IFN expression in the gastrointestinal area, but in an amount less than sufficient to significantly alter systemic IFN.

2. The method of claim 1 wherein the TLR agonist is: 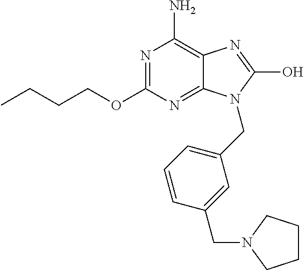 [SMILES: CCCCOc1nc(N)c2nc(O)n(Cc3cccc(CN4CCCC4)c3)c2n1]
6-amino-2-butoxy-9-(3-(pyrrolidin-1-ylmethyl)benzyl)-9H-purin-8-ol ; or 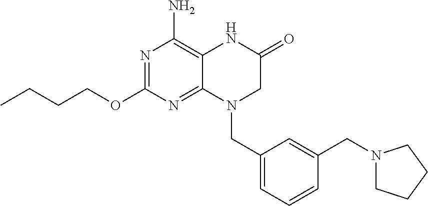 [SMILES: CCCCOc1nc(N)c2c(n1)N(Cc1cccc(CN3CCCC3)c1)CC(=O)N2]
4-amino-2-butoxy-8-(3-(pyrrolidin-1-ylmethyl)benzyl)-7,8-dihydropteridin-6(5H)-one;

3. The method of claim 1 wherein the gastrointestinal disorder is cancer or a pathogen infection.

4. The method of claim 3 wherein the cancer is hepatocellular cancer, colorectal cancer, gastrinoma, insulinoma, glucagonoma, pancreatic ductal adenocarcinoma, VIPoma, somatostatinoma, ACTHoma, adenocarcinoma of the stomach, leiomyosarcoma or adenomatous polyposis.

5. The method of claim 3 wherein the pathogen infection is a parasitic infection.

6. The method of claim 5 wherein the parasitic infection is: Clonorchis sinensis, Opisthorchis felineus, Opisthorchis viverrini, Dicrocoelium dendriticum, Elaeophora elaphi, Fasciola, Plasmodium, Amoebiasis, Pseudosuccinea columella, Schistosoma mansoni, Visceral leishmaniasis, Histomonas meleagridis, Histomoniasis, Echinococcus multilocularis, Fasciolosis, Schistosomiasis, Capillaria hepatica, Prosthogonimidae, Alveolar hydatid disease, Clonorchiasis, Toxoplasmosis or Opisthorchiasis.

7. The method of claim 3 wherein the pathogen infection is a fungal infection.

8. The method of claim 7 wherein the fungal infection is histoplasmosis, coccidiodomycosis, North American blastomycosis or cryptococcosis.

9. The method of claim 3 wherein the pathogen infection is a bacterial infection.

10. The method of claim 3 wherein the pathogen infection is a viral infection.

11. The method of claim 10 wherein the viral infection is hepatitis B.

12. The method of claim 10 wherein the viral infection is hepatitis C.

13. The method of claim 10 wherein the viral infection is HIV.

14. The method of claim 1 wherein the gastrointestinal disorder is a food allergy.

15. The method of claim 14 wherein the food allergy is a peanut allergy.

16. The method of claim 1 wherein the gastrointestinal disorder is an autoimmune disorder.

17. The method of claim 16 wherein the autoimmune disorder is Crohn's disease.

18. The method of claim 2 wherein the TLR agonist is: 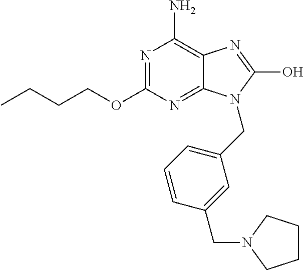 [SMILES: CCCCOc1nc(N)c2nc(O)n(Cc3cccc(CN4CCCC4)c3)c2n1]
6-amino-2-butoxy-9-(3-(pyrrolidin-1-ylmethyl)benzyl)-9H-purin-8-ol

19. The method of claim 2 wherein the TLR agonist is

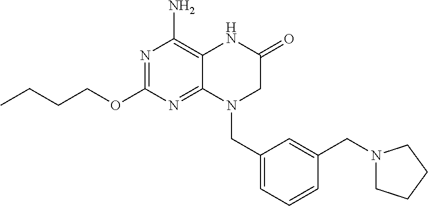 [SMILES: CCCCOc1nc(N)c2c(n1)N(Cc1cccc(CN3CCCC3)c1)CC(=O)N2]

20. The method of claim 19, wherein said TLR agonist is used in combination with another active pharmaceutical ingredient.

21. The method of claim 11 wherein the TLR agonist is: 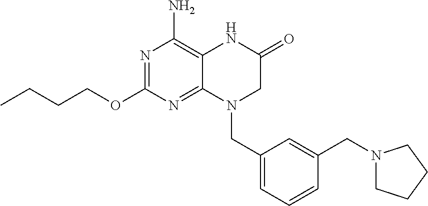 [SMILES: CCCCOc1nc(N)c2c(n1)N(Cc1cccc(CN3CCCC3)c1)CC(=O)N2]
Compound B.

22. The method of claim 21 wherein compound B is administered to a patient in need thereof, in a total dose of less than 12 mg twice per week.

23. The method of claim 21 wherein compound B is administered once or twice per week.

24. The method of claim 23 wherein compound B is administered once per week.

25. The method of claim 22 wherein compound B is administered in combination with a nucleoside reverse transcriptase inhibitor; a non-nucleoside reverse transcriptase inhibitor; a protease inhibitor; a cyclophilin inhibitors; immune modulators; or a combination thereof.

26. The method of claim 22 wherein compound B is administered in combination with a product selected from: etbecavir, telbivudine, lamisvudine, adofovir dipivoxil, entecavir, tenofovir disoproxil fumarate, emtricitabine, tenofovir dipivoxil or its salts and co-crystals; or a yeast-based therapeutic vaccination; or combinations thereof.

27. A kit comprising an oral dosage form pharmaceutical composition of compound B, in a package adapted for distribution of said oral dosage form pharmaceutical composition in an amount between 0.5 mg and 12 mg. once per week.
